(19) United States
(12) Patent Application Publication
(10) Pub. No.: US 2010/0019918 A1
(43) Pub. Date: Jan. 28, 2010

(54) Probe Insertion Guide with User-Directing Features

(75) Inventors: Ofer Avital, Yokneam Ilit (IL); Eyal Kochavi, Haifa (IL); Amir Pansky, Atlit (IL); Yaron Hefetz, Herzlia (IL); Pazit Pianka, Kochav Yair (IL)

Correspondence Address: MARTIN D. MOYNIHAN d/b/a PRTSI, INC., P.O. BOX 16446, ARLINGTON, VA, 22215, US

(73) Assignee: Galil Medical Ltd., Yokneam (IL)

(21) Appl. No.: 12/226,948

(22) PCT Filed: May 2, 2007
(86) PCT No.: PCT/IL2007/000538

§ 371 Date: Aug. 26, 2009

Related U.S. Application Data
(60) Provisional application No. 60/796,519, filed on May 2, 2006.

(51) Int. Cl. G08B 21/00; A61B 18/02

(52) U.S. Cl. 340/686.4; 606/20

(57) ABSTRACT

The present invention relates to a probe-insertion guiding apparatus for guiding a user towards correct insertion of therapeutic probes into a patient. The apparatus comprises a template which comprises a plurality of apertures sized to accommodate therapeutic probes and to guide insertion of such probes into a body, a probe command receiver for receiving probe insertion commands specifying template apertures through which probes are to be inserted, and at least one of a) a sensory output device operable to indicate to a user which aperture is to receive a next inserted probe, and b) a probe detection device operable to detect insertion of a probe into an aperture. Preferred embodiments include feedback modules providing feedback to a user indicating whether the user's probe did or did not correspond to a probe insertion specified by a received probe insertion command.

### RELATED APPLICATIONS

This Application claims the benefit under 119(e) of U.S. Provisional Patent Application No. 60/796,519 filed May 2, 2006, the contents of which are incorporated herein by reference.

This application is related to U.S. application Ser. No. 11/219,648, the disclosure of which is incorporated herein by reference.

This application is related to two other PCT applications being filed on even date with this application in the Israel Receiving Office having the titles CRYOTHERAPY PLANNING AND CONTROL SYSTEM and CRYOTHERAPY INSERTION SYSTEM AND METHOD, and Attorney docket Nos. 33982 and 39262, and sharing applicant Galil Medical Ltd. with this Application, the disclosures of which are incorporated herein by reference.

### FIELD AND BACKGROUND OF THE INVENTION

The present invention relates to devices and methods for guiding insertion of treatment probes into a body of a patient. More particularly, the present invention relates to use of a probe insertion template apparatus having features which direct user actions and which respond to user actions during a probe-insertion process.

Use of a probe template to control and direct insertion of a plurality of therapeutic probes into a body is well known in a variety of surgical disciplines. In the field of brachytherapy, for example, templates have been used to guide insertion of a plurality of probes delivering radioactive elements to a therapeutic target such as a prostate. In the field of cryosurgery, templates have similarly been used to guide delivery of a plurality of probes towards and into an organic target, enabling to perform cryoablation of a large target in a pre-planned and organized manner.

One example of such a use is presented by U.S. Pat. No. 6,142,991 to Schatzberger. Schatzberger teaches what he calls a “high resolution” cryosurgical method and device for treating a patient's prostate. Schatzberger's “high resolution” method comprises the steps of (a) introducing a plurality of cryosurgical probes to the prostate, the probes having a substantially small diameter and are distributed across the prostate, so as to form an outer arrangement of probes adjacent the periphery of the prostate and an inner arrangement of probes adjacent the prostatic urethra; and (b) producing an ice-ball at the end of each of said cryosurgical probes, so as to locally freeze a tissue segment of the prostate. Distribution of this plurality of cryosurgical probes, and the consequent planned distribution of ice-balls resulting from operating the distributed probes, is accomplished through use of Schatzberger's apparatus, which comprises (a) a plurality of cryosurgical probes of small diameter, the probes serve for insertion into the patient's organ, the probes being for producing ice-balls for locally freezing selected portions of the organ; (b) a guiding element (which corresponds to an element referred to herein as a “template”) including a net of apertures for inserting the cryosurgical probes therethrough; and (c) an imaging device for providing a set of images, the images being for providing information on specific planes located at specific depths within the organ, each of said images including a net of marks being correlated to the net of apertures of the guiding element, wherein the marks represent the locations of ice-balls which may be formed by the cryosurgical probes when introduced through said apertures of the guiding element to said distinct depths within the organ.

Schatzberger's devices and method provide the advantages of high resolution of treatment along the axis of penetration of the cryosurgical probe into the patient's organ as well as along the planes perpendicular to the axis of penetration, thereby enabling to effectively destroy selective portions of a patient's tissue while minimizing damage to adjacent tissues and organs, and to selectively treat various portions of the tissue located at different depths of the organ, thereby effectively freezing selected portions of the tissue while avoiding the damaging of other tissues and organs located at other depth along the axis of penetration.

U.S. Pat. No. 6,206,832 to Downey et al provides an additional example of use of a template for guiding placement of one or more medical instruments into a target tissue during a minimally invasive medical procedure. Downey's apparatus comprises a template (which Downey calls a “reference means”) having a plurality of apertures arranged in a predefined manner which are sized to permit at least one medical instrument to pass therethrough. Downey's apparatus further comprises a processing means in communication with an ultrasonographic system, and a mounting means for mounting the reference means in a predetermined relationship to an ultrasonographic transducer. Downey's processing means determines the spatial relationship between the target tissue and the template and further merges a representation of the plurality of template apertures with an ultrasonographic image to form a positioning image. Downey's positioning image assists in guiding and placement of one or more medical instruments into a target location by identifying a path to the target location via a selected aperture.

Thus, use of templates for guiding placement of therapeutic probes within target tissues of a patient according to Schatzberger's teachings, according to Downey's teachings, and indeed according to what has become standard surgical practice in a variety of minimally invasive surgical disciplines, involves selection of one or, typically, a plurality of selected template apertures from among a larger plurality of apertures present within a template, and insertion of one or many therapeutic probes through said selected template apertures, generally to pre-determined desired depths of penetration, for purposes of using probes so inserted to perform therapeutic acts such as installation of radioactive brachytherapy “seeds” or creation of cryosurgical ice-balls to affect cryoablation of tissues.

Selection of template apertures may be made by a surgeon. However, in advanced systems template aperture selection may be made by computational algorithms operating within a processor-based control system, according to calculations based on patient and target information which is either input by an operator, or gleaned algorithmically from data collected by visualization modalities, or both. Downey, for example, teaches a system for facilitating selection of apertures by an operator, by combining images of the template apertures with ultrasound images of target tissues. U.S. patent application Ser. No. 11/219,648, and U.S. provisional application 60/796,519, both of which are incorporated herein by reference, teach systems whereby algorithmic methods are used by processor-based controllers to recommend for use a selected set of apertures, and to recommend selected insertion depths for probes in each selected aperture.

It is, however, a disadvantage in all such prior-art template-based systems that, once a set of apertures has been selected by a surgeon and/or recommended by a processor-based algorithmic recommender system, probes must be manually inserted by a surgeon through the selected template apertures and into a patient.

Field experience with template systems has demonstrated that the process of insertion of probes through selected apertures and into a patient is somewhat time-consuming and tends to be somewhat prone to errors. Inefficient or time-consuming probe placement processes are wasteful of the expensive time of medical personnel and equipment and increase patient discomfort, and uncorrected errors in probe placement can significant endanger to patient health and delay recovery. In particular, when probe placement is used for used for ablation of malignant tissue, incorrect placement of therapeutic probes can cause partially ineffective ablation, leading to proliferation of malignant cells. More generally, incorrect placement of therapeutic probes use for ablation can cause damage or destruction of healthy tissues not intended to be ablated, leading to damage of important systems and structures located near ablation targets, and causing serious deleterious effects to patient post-operative health, delaying recovery, and reducing subsequent patient quality of life.

There is a need for, and it would be highly desirable to have, devices and methods for introducing a plurality of probes through a template into target tissues within a body, optionally absent some or all these disadvantages.

### SUMMARY OF THE INVENTION

The present invention relates to methods and devices for facilitating correct insertion of therapeutic probes into a patient through a probe-guiding template.

Preferred embodiments include a command-reception module for receiving probe placement commands, a probe-guiding apparatus comprising a template with an array of apertures for guiding therapeutic probes into a target, and further comprising visual and/or auditory signaling modules on or near the template, which modules are operable to provide visual and/or auditory cues directing a user towards correct insertion of probes through the template according to received probe insertion commands. Preferred embodiments additionally include sensors for detecting actual probe insertions real time, and signaling modules operable to provide visual and/or auditory feedback to a user, indicating whether actual probe insertions performed by a user are correctly correspond to probe placement commands received by the command-reception module. Further preferred embodiments include a processor-based control module for generating probe placement commands receivable by the command-reception module, which commands are generated by algorithmic computations based on information provided by a user and/or based on information gleaned from user-input or automated analysis of images provided by imaging modalities such as ultrasound, x-ray, fluoroscopy, MRI, or other imaging modalities. Further preferred embodiments comprise probe-depth sensors operable to determine and to report the depth to which a probe has been inserted through a template aperture, and feedback modules operable to provide visual or auditory feedback to a user relating actual insertion depth of a probe to insertion-depth commands received by the command-reception module. Feedback provided by the various feedback modules may be provided directly, by real-time production of sounds, lights, images or other immediate sensory feedback, or may be provided to a processor-based device and/or memory device operable to store the feedback information for relay to a distant user or computer or for delayed or remote analysis.

User-directing signaling elements include LED lighting elements and directed-beam lighting elements which may be positioned on or near the template, tonal auditory signaling indicating that a probe approaching a template is (or is not) appropriately positioned for a desired insertion, and vocal instructions, by computer generated voice and/or by selection of pre-recorded instructions, instructing a user where to insert a probe. Optionally, a user can indicate to the system that a probe is placed (and/or correctly placed) and that a next probe location should be indicated to the user. Optionally, a dedicated hardware button is provided. Alternatively or additionally, a GUI interface (e.g., interface 54) is used.

Detectors usable for detecting probe insertions include magnetic detection sensors, electrical sensors such as micro-switches switched by presence of a probe in an aperture and circuit-breaking electrical contacts positioned so that an inserted probe completes a circuit, and light-beam detection sensors so positioned that a light beam is interrupted, and that interruption detected by a light sensor, when a probe is inserted in a particular aperture. Detectors in preferred embodiments may also include simple video camera lenses producing images of a template, coupled with image interpretation software operable to determine which apertures of a template contain, and which do not contain, inserted probes.

Templates of the present invention may be sterilizable-reuseable, or may be designed for one-time use. To facilitate template sterilization and/or to render one-time templates relatively inexpensive, signaling and/or sensor elements may be mounted on a template frame designed for holding a sterile template in a standard position, bringing template apertures into a standardized positional relationship with sensor and/or signaling elements positioned on that frame.

In a preferred embodiment of the present invention, feedback is further provided in the form of a safety cut-off module connected to the probe activation systems and operable to warn and operator and/or prevent probe operation (e.g. cooling of inserted cryoprobes) if an actual probe insertion pattern does not correspond to a probe insertion pattern designated by received probe insertion commands.

The present invention, in some embodiments thereof, successfully addresses the shortcomings of the presently known configurations by providing devices and methods facilitating correct and rapid insertion of a plurality of treatment probes through a template and into a patient in a pre-determined user-selected or algorithmically selected pattern, said insertions being to prescribed insertion depths, thereby facilitating and speeding an important and time-consuming portions of surgical procedures while minimizing or preventing probe insertion errors, thereby saving time for doctor and patient and safeguarding patient health and well-being.

There is thus provided in accordance with an exemplary embodiment of the invention, a probe-insertion guiding apparatus comprising:

(a) a template which comprises a plurality of apertures sized and shaped to accommodate therapeutic probes inserted therethrough;

(b) a command receiver operable to receive a command specifying a template aperture through which a probe is to be inserted; and

(c) at least one of group consisting of:
(i) a first output device operable to output a signal serving to indicate which template aperture is specified by said received command; and
(ii) a probe detection module operable to detect when and where a probe is inserted in one of said plurality of apertures.

Optionally, the apparatus comprises said first output device, and wherein said output signal is sensory signal perceivable by a user.

In an exemplary embodiment of the invention, the apparatus comprises said first output device, and wherein said output signal is an electronic signal operable to control a servomechanism. Optionally, said electronically generated signal is a digital signal. Alternatively, said electronic signal is an analog signal.

In an exemplary embodiment of the invention, said electronic signal is communicated by a means selected from a group consisting of wired communication, wireless radio communication, optical communication, and infra-red communication.

In an exemplary embodiment of the invention, the apparatus comprises both said first output device and said probe detection module.

In an exemplary embodiment of the invention, the apparatus comprises a feedback mechanism operable to inform a user whether or not a probe inserted by said user has been inserted in an aperture specified by a received command.

In an exemplary embodiment of the invention, the apparatus comprises said first output device, and wherein said output device comprises at least one of a group consisting of:
(a) a lighting device operable to highlight a selected aperture;
(b) a pair of lighting devices operable to specify a selected aperture by highlighting a specific row of apertures and a specific column of apertures;
(c) a pair of light beams operable to highlight a portion of a probe positioned near or within a selected aperture;
(d) a LED highlighting module operable to indicate a selected aperture by illuminating selected LEDs, thereby creating a visual pattern which comprises two illuminated lines intersecting at said selected aperture;
(e) an auditory output device operable to emit a characteristic sound when a probe approaches a selected aperture;
(f) a voice output device operable to verbally identify a selected aperture; and
(g) an image output device operable to present to a user an image of said template on which an image of a selected aperture is highlighted.

Optionally, the apparatus comprises a lighting device operable to use colored illumination to highlight a selected aperture.

In an exemplary embodiment of the invention, the apparatus comprises said probe detection device, and wherein said probe detection device comprises at least one of a group consisting of:
(a) a magnetic detector;
(b) a microswitch;
(c) a current detector operable to detect a current in a circuit completed by passage of a probe body between electrical contacts;
(d) a voltage detector operable to detect a voltage in a circuit completed by passage of a probe body between electrical contacts; and
(e) an image-interpretation detector which comprises
   (i) a camera operable create a real-time image of said template; and
   (ii) image interpretation software operable to detect presence of a probe in or near a selected aperture by algorithmic interpretation of a real-time template image created by said camera.

In an exemplary embodiment of the invention, the apparatus comprises a feedback module which comprises a second output device and a controller operable to receive input from said probe detection device and to command said second output device to output a pre-defined first signal when said probe detection device reports insertion of a probe in an aperture specified by said received command. Optionally, said first output device and said second output device are a common output device.

In an exemplary embodiment of the invention, the apparatus comprises a feedback module which comprises a second output device and a controller operable to receive input from said probe detection device and to command said second output device to output a pre-defined second signal when said probe detection device reports insertion of a probe in an aperture different from that specified by said received command.

In an exemplary embodiment of the invention, said controller is further operable to command said second output device to output a pre-defined second signal when said probe detection device reports insertion of a probe in an aperture different from that specified by said received command.

In an exemplary embodiment of the invention, said second output device comprises one or more of a group consisting of:
(a) a generator of light;
(b) a generator of sound;
(c) a voice generator;

(d) an image display;
(e) an electronic signal generator operable to generate a signal detectable by a remote electronic receiver; and
(f) a control-signal generator operable to generate a signal directed to a remote controller operable to control activation of an inserted probe.

In an exemplary embodiment of the invention, the apparatus comprises at least one depth detector operable to detect depth of insertion of a probe within an aperture. Optionally, said depth detector is an optical reader operable to count markings on an inserted probe as said probe is inserted in an aperture. Optionally, the apparatus is further operable to provide feedback to a user, which feedback provides information relating actual inserted depth of said inserted probe to a probe depth command received by said command receiver.

In an exemplary embodiment of the invention, the apparatus comprises a reusable frame to which said template is attachable, said frame and said template being so shaped and so positioned when said template is attached to said frame that a probe may traverse said apertures of said template while said template is attached to said frame. Optionally, said frame comprises at least one electrical component. Alternatively or additionally, said template is designed for one-time use.

There is also provided in accordance with an exemplary embodiment of the invention, a method of guiding probe insertion by a user, comprising:
(a) indicating a next probe location to a user;
(b) detecting an indication that a probe was inserted into the location.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a therapeutic probe usage system which comprises a probe insertion guidance apparatus, according to an embodiment of the present invention;

FIG. 2 is a simplified schematic of a probe insertion template connected to an ultrasonic probe, according to methods of prior art;

FIG. 3 is a simplified schematic of a template operable to use light patterns provided by LEDs or similar light sources to draw user attention to an aperture designated by a command, according to an embodiment of the present invention;

FIG. 4 is a simplified schematic of a template utilizing pairs of lights to indicate row and column of a selected aperture, according to an embodiment of the present invention;

FIG. 5 is a simplified schematic of a template attached to a frame, according to an embodiment of the present invention;

FIG. 6 is a simplified schematic of a template/frame combination utilizing a template of simplified construction, according to an embodiment of the present invention; and

FIG. 7 is a simplified schematic providing a composite view of a template comprising a variety of output devices and probe detection sensors, according to an embodiment of the present invention.

### DESCRIPTION OF THE PREFERRED EMBODIMENTS

The present invention relates to devices and methods for facilitating correct insertion of a plurality of therapeutic probes into body tissues. Specifically, the present invention can be used to guide a surgeon during insertion of a plurality of cryoprobes through a probe-guiding template. The apparatus described receives probe-insertion commands, provides visual and auditory cues to a surgeon to facilitate his insertion of probes through template apertures according to those commands, and provides visual and auditory feedback informing inform the surgeon whether his probe insertions conform to the received probe-insertion commands. Thus the present invention serves to facilitate and speed probe insertion, while safeguarding against probe insertion errors.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

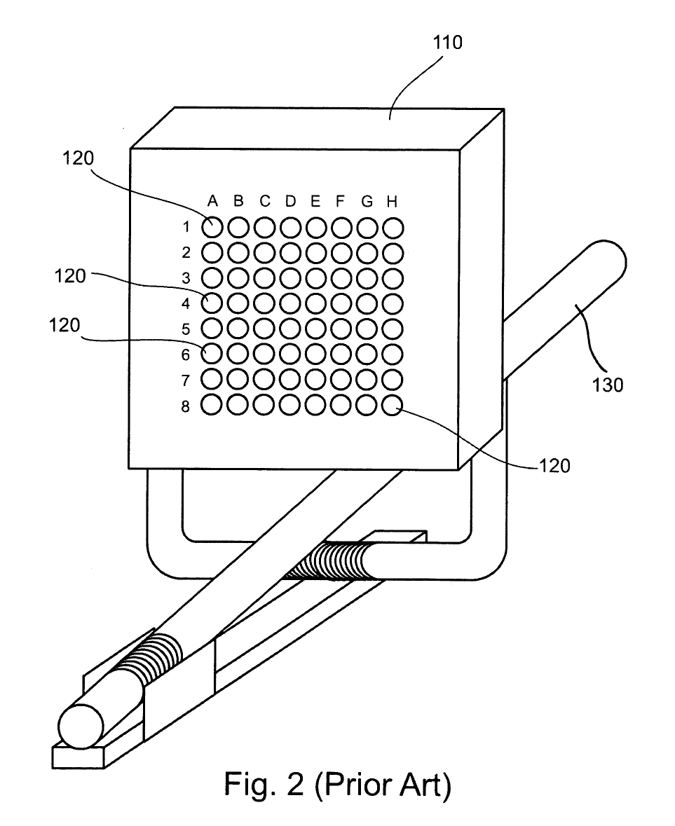

In the following, the terms “template” and “probe-insertion template” are used to refer to any apparatus comprising a plurality of apertures sized to accommodate and to direct insertion of one or more therapeutic probes into a body. In current use, “templates” are typically rectangular objects constructed of metal or plastic and comprising a regular two-dimensional array of apertures standard size and parallel orientation. Such a template is presented, by way of example, in FIG. 2, and is discussed hereinbelow. It is to be understood, however, that the template examples provided by FIG. 2 and by the following Figures are exemplary only, and that the term “template” as used herein is not limited to any particular form or method of construction, nor is that term limited to the examples provided by the Figures. As used herein, a “template” is any object comprising a plurality of apertures through which one or more therapeutic probes may be inserted, the apertures serving to direct or limit insertion direction and/or depth of insertion of probes inserted through the apertures.

In the following, the terms “therapeutic probe” and “therapeutic probes” are used to describe the probes inserted through a template and into a patient. These terms are to be understood to refer to any medically useful device which may be inserted into a body of a patient or animal through a template. In particular, the terms “therapeutic probe” and “therapeutic probes” are to be understood to include cryoprobes, brachytherapy probes, thermal sensors, RF probes, protective heating probes, thermal ablation probes of any type, and any other type of probe which can appropriately be introduced into a body through a template.

It is expected that during the life of this patent many relevant therapeutic probes and probe-insertion templates will be developed, and the scope of the terms “therapeutic probe” and “template” is intended to include all such new technologies a priori.

As used herein the terms “about” and “approximately” refer to ±20%.

In discussion of the various figures described hereinbelow, like numbers refer to like parts. The drawings are generally not to scale

For clarity, non-essential elements are omitted from some of the drawings.

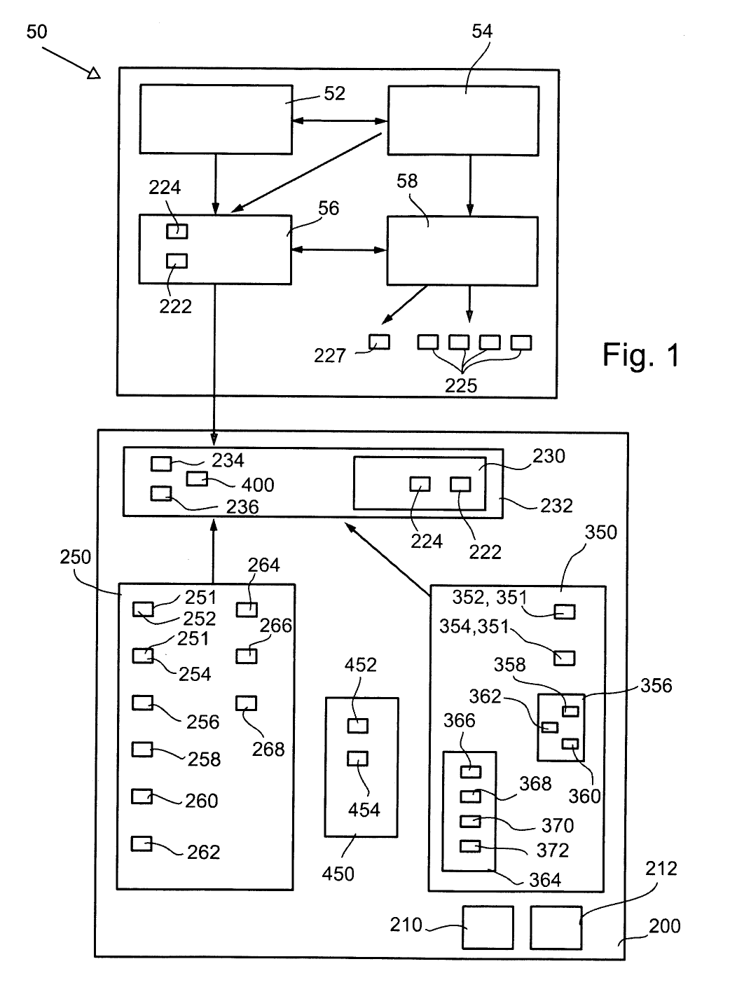

Attention is now drawn to FIG. 1, which is a simplified schematic of a therapeutic probe usage system which comprises a probe insertion guidance apparatus, according to an embodiment of the present invention.

FIG. 1 presents a probe usage system used with a probe insertion guidance apparatus 200. Usage system 50 may be any system used to operate and control one or more therapeutic probes 225 introduced into the body for therapeutic or investigative purposes. In a simplified exemplary embodiment of usage system 50 presented in FIG. 1, imaging information sources 52 and user interface 54 both provide input information to a probe insertion planner 56, and to a real-time probe activation controller 58. A servomechanism 227 may be provided for moving and manipulating probes 225.

Imaging information sources 52 may be any combination of imaging modalities. In typical probe usage systems, imaging sources 52 may comprise MRI equipment, ultrasound probes operated within or without the body, fluoroscopic scans, CT scanner or other x-ray equipment, television cameras and optical scopes, and any other similar imaging modalities. Imaging sources 52 may comprise real-time images created during probe use and/or may comprise preliminary images created prior to surgery and in anticipation thereof. Imaging sources 52 may further comprise an image combining module operable to combine two or more images into a composite image, and may include computation modules for registering a plurality of different images according to a common scale and common set of spatial coordinates.

Typically, images created by imaging sources 52 are presented to a user by user interface 54. User interface 54 may comprise any interface equipment operable to present images to a user and receive user input. Typically, user interface 54 will enable a user to characterize portions of displayed images (e.g. to identify treatment targets within an image, or to specify desired probe insertion positions with respect to a displayed image). User interface 54 will also typically enable a user to input command decisions or preferences, such as the number and type of probes to be used, their desired operating parameters, and other similar operating details.

Probe insertion planner 56 is an optional module, present for example in Schatzberger's system described in the background section hereinabove. Planning module 56, if present, serves to calculate one or more probe insertion positions appropriate for treating a treatment target, as that treatment target is detected in images provided by imaging sources 52 and/or is specified by a surgeon through user interface 54. If planning module 56 is absent, a probe insertion plan may simply be specified by a surgeon using user interface 54.

A probe insertion plan, whether created by planning module 56 or input directly by a surgeon, comprises a set of template aperture specifications 222 and optionally also probe depth specifications 224. These specifications (also referred to herein as “commands”) refer to apertures 220 within a template 210, which is described in detail hereinbelow.

Prior to presenting template 210, attention is first drawn to FIG. 2, which is a simplified schematic of a probe insertion template 110 connected to an ultrasonic probe, according to methods of prior art. FIG. 2 presents a template 110 having an array of apertures 120, each aperture sized to accommodate and direct a therapeutic probe insertable therethough. The exemplary prior-art template presented in FIG. 2 is designed to facilitate insertion of therapeutic probes into a prostate while the insertion process is observed on an ultrasound display displaying images generated by an ultrasound probe 130 inserted into a patient's rectum. Apertures 120 of template 110 are designed to direct a plurality of probes inserted therethrough into body tissues along substantially parallel paths. It is to be noted, however, that the template structure presented by FIG. 2, while typical, is not to be understood as limiting. Templates contemplated by the present invention may be of any shape and may comprise non-regular aperture arrays and non-parallel apertures.

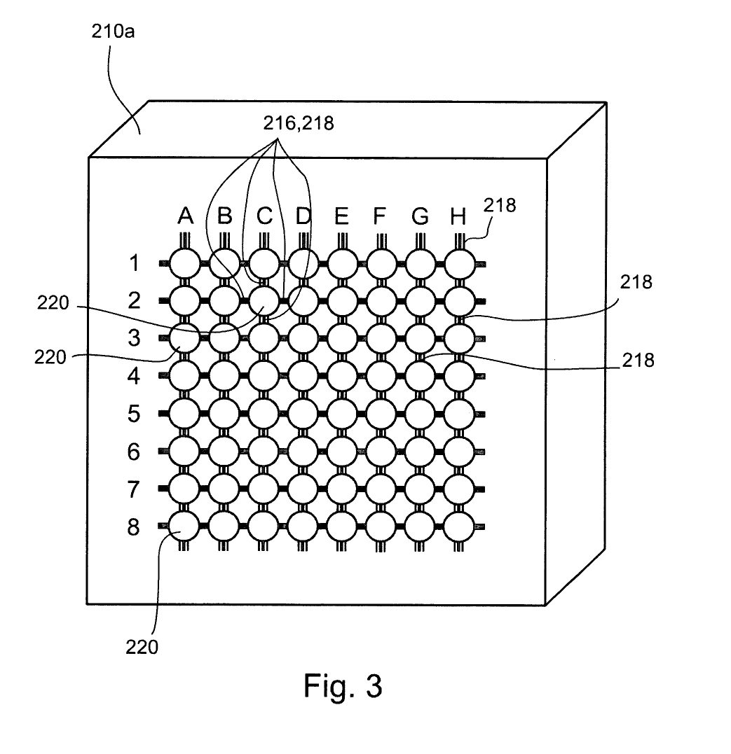

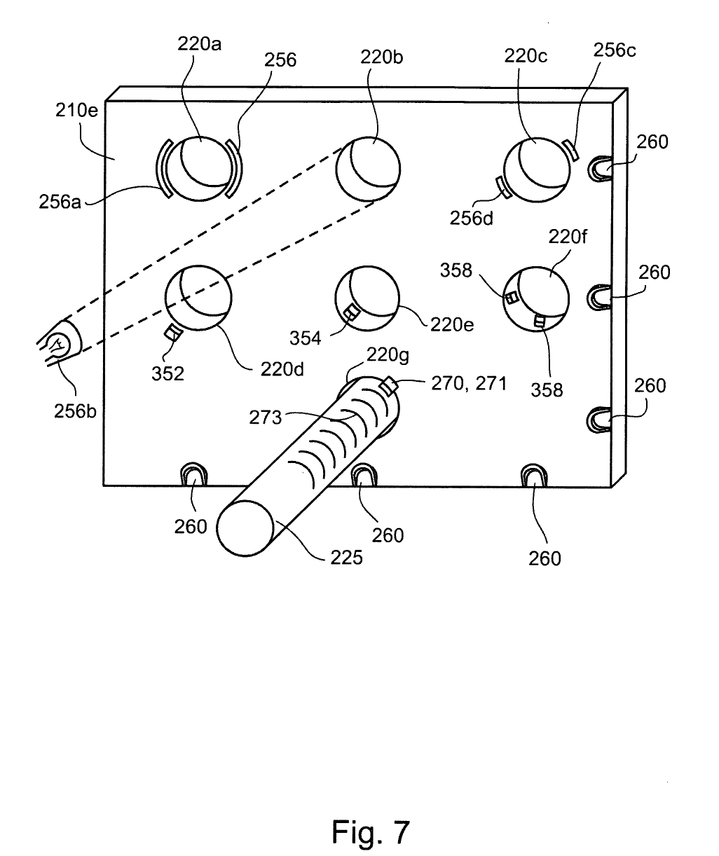

Returning attention now to FIG. 1, Figure further presents a probe insertion guidance apparatus 200. Probe insertion guidance apparatus 200 comprises a template 210 which comprises a plurality of apertures 220 sized and shaped to accommodate therapeutic probes 225 inserted therethrough. (Apertures 220 are shown in FIGS. 3-7, probe 225 is shown in FIG. 7). Apparatus 200 further comprises a guidance controller 232 which comprises a command receiver 230 and preferably comprises a programmable processor 234 and a memory 236. Command receiver 230 is operable to receive commands 222 each specifying a template aperture 220 through which a probe 225 is to be inserted. Optionally, guidance controller 232 may be integrated within a multi-task processor, for example the processor executing planning module 56. Thus, command receiver 230 is operable to receive one or more command specifications 222 such as may be provided by a planning module 56 and/or a surgeon using user interface 54. Commands receivable by command receiver 230 may alternatively or additionally include probe depth specifications 224 specifying a depth to which a probe 225 is to penetrate into and through an aperture 220. In some embodiments, the specifications are of ranges of allowed and/or desired placement. Optionally, the system generates a warning if these ranges are passed (e.g., a range may be defined to prevent puncturing of the urethra). Optionally, the template includes an actuator adapted to extend into an aperture and block and/or lock a probe form advancing too far or in a wrong location. Optionally, the sensor doubles as an actuator, for example, if the sensor/actuator is a linear magnetic actuator which doubles as a translation sensor indicating presence in the aperture.

In a preferred embodiment of the present invention, apparatus 200 further comprises an output device module 250 which comprises one or more output devices 251, which output devices are operable to provide sensory signals perceivable by a user and/or electronic signals readable by, and operable to control, servomechanism 227, said output signals serving to indicate which template aperture 220 is specified by said received command. Output devices 250 may thus include digital signal emitters 252 operable to communicate by wire, by wireless radio communication, by optical signal, by infrared interface, or by any similar communication means. Output devices 250 may further or alternatively include analog signals emitter 254, a plurality of lighting devices 256 each operable to highlight a selected aperture either by producing light near said selected aperture or by shining light towards said aperture or by using colored lighting to change the apparent color of an aperture or its neighborhood, lighting devices 258 operable to specify a selected aperture when illuminated in pairs, one light of the pair of lights highlighting a specific row of apertures and one light of the pair of lights highlighting a specific column of apertures, lighting devices 260 also operable to specify a selected aperture when illuminated in pairs, lighting devices 260 being light beam projection devices aimed so as to illuminate a probe when that probe is placed in position to be inserted into that selected aperture (or simply when that probe is positioned near the selected aperture), a LED highlighting module 262 operable to highlight a selected aperture by illuminating LED which create a visual pattern which points toward the selected aperture or which appears as at least two illuminated lines intersecting at said selected aperture, an auditory output device 264 operable to emit a characteristic sound when a probe approaches a selected aperture, a voice output device 266 operable to verbally identify a selected aperture, and an image output device 268 operable to present to a user an image of template 210 on which an image of a selected aperture 220 is highlighted.

In a further preferred embodiment of the present invention, apparatus 200 further comprises a probe detection module 350 operable to detect when and where a probe is inserted in an aperture 220. In some embodiments, a polling system is used to poll the apertures, so the resolution of “when” may be arbitrarily reduced. Detection module 350 comprises one or more detectors (sensors) 351 or combinations of sensors 351 operable to detect instances of probe insertion within an aperture 220 and preferably operable to detect instances of approach of a probe 225 near to an aperture 220. In particular, detection module 350 may comprise one or more of the following: a magnetic detector 352 or preferably a plurality of magnetic detectors 352 each mounted within or near an aperture 220, a microswitch 354 or preferably a plurality of microswitches 354, each preferably mounted within an aperture 220 and operable to be switched by pressure of a probe positioned within (i.e. traversing or partially traversing) that aperture 220, an electrical detector 356 comprising a set of contacts 358 (one of which may be the body of template 210) connected to a power source and a current detector 360 or a voltage detector 362 and operable to complete a circuit when a body of a metallic probe is positioned within an aperture 220, and an image-interpretation detector 364 which comprises one or more cameras 366 operable to create an image 368 of template 210 in real time and a processor 370 running image interpretation software 372 operable to detect presence of a probe in or near a selected aperture 220 by algorithmic interpretation of image 368 created by camera 366.

Apparatus 200 may comprise either output devices 250 or probe detection module 350, yet in a preferred embodiment apparatus 200 comprises both one or more output devices 250 and also a probe detection module 350. In a particularly preferred embodiment, apparatus 200 comprises both output devices 250 and probe detection module 350, and further comprises a feedback mechanism 400, preferably implemented as a set of programmed instructions in guidance controller 232. Feedback mechanism 400 uses probe detection module 350 to detect probe insertions into apertures 220 and to determine into which aperture 220 a probe 225 has been inserted. Mechanism 400 then compares the identity of an aperture 220 into which a probe has been inserted with the aperture identity specified in an aperture specification 222 (also referred to herein as a “probe insertion command 222”). Mechanism 400 is thus enabled to determine whether a detected probe insertion event is correct or incorrect with respect to the probe insertion specified by the current probe insertion command 222. In a preferred embodiment, feedback mechanism 400 utilizes some of or all of output devices 250 to provide feedback to a user inserting a probe, the feedback signal provided serving to inform the user whether his insertion was “correct” (i.e. according to the insertion command 222) or “incorrect” (i.e. differing from the current insertion command).

Output devices used by mechanism 400 may be output devices 250 also used to direct a user where to insert a probe, as described hereinabove, or may alternatively be other output devices. In a preferred embodiment feedback mechanism 400 is characterized in that controller 232, under command of mechanism 400 software, commands a feedback output device 450 to output a characteristic first signal 452 when probe detection device 350 reports insertion of a probe in an aperture specified by a received command 222, and commands feedback output device 450 to output a characteristic second signal 454 when probe detection device 350 reports insertion of a probe in an aperture different from that specified by a received command 232.

Feedback output device 450 may be similar to (or identical to) output devices 250, and indeed same devices may be used for both purposes. Thus feedback output device 450 may comprise a generator of light, a generator of sound, a voice generator speaking computer-generated voice patterns and/or reproducing recorded words or phrase. Feedback output device 450 may further comprise an image display which may, for example, portray command aperture selection and actual aperture insertion in contrasting colors or with a synthesized animation dramatizing a mistaken insertion.

Feedback device 450 may also provide an electronic signal detectable by a remote electronic receiver. In a particularly preferred embodiment, an electronic signal generated by feedback device 450 and indicating an insertion point different from an insertion point selected in a received insertion command may be transmitted to probe actuation controller 58 which, as part of probe usage system 50, controls actuation of probes 225 inserted through template 210 and into a patient. Thus, for example, if probes 225 are cryoprobes 226 serving to cryoablated body tissues, actuation controller 58 may be programmed to prevent actuation of probes 226 and suspend cryoablation until a detected probe insertion error is corrected (or “misplacement” of the probe is authorized) by a user.

Attention is now drawn to FIG. 3, which is a simplified schematic of a template 210a wherein light patterns 262 provided by LEDs or similar light sources may be used to draw user attention to an aperture designated by a command, according to an embodiment of the present invention. Template 210a comprises selectively lightable LED lights 218 between adjacent apertures. Patterns of illumination may be used to designate a selected aperture 210. For example, LEDs marked 216 surrounding aperture C2 might be used to call user attention to aperture C2. In another example, all LEDs in column F and all LEDS in row 6 might be lit to call user attention to aperture F6, by presenting an image of vertical and horizontal lit lines intersecting at aperture F6. In a preferred embodiment comprising probe detection sensors 350, insertion of a probe into a “correct” aperture could be followed by extinguishing of let LEDs designating that successful selection, perhaps together with an audible rising tone indicating successful insertion, perhaps followed by lighting of LEDs designating an additional insertion site. Incorrect probe insertion detected by probe detection sensors 350, on the other hand, might be followed by, say, a louder descending audible tone and a flashing of LEDS around the mis-inserted probe, which flashing would continue until the mis-inserted probe was removed or until an override command by the user indicates that the “mis-inserted” probe is to be allowed to remain in place.

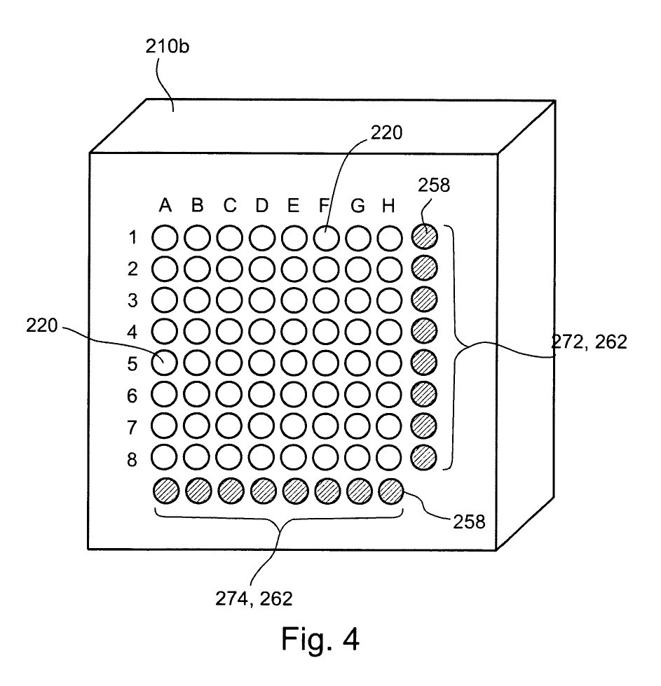

Attention is now drawn to FIG. 4, which is a simplified schematic of a template 210b utilizing pairs of lights to indicate row and column of a selected aperture, according to an embodiment of the present invention. FIG. 4 presents a detailed view of lighting devices 258. Alongside a rectangular array of apertures 220 a vertical row of lighting elements 272 and a horizontal row of lighting elements 274 are provided. According to the embodiment of FIG. 3, controller 232 can designate a selected aperture by lighting one lighting element from among vertical row elements 272 and lighting one element from among horizontal row elements 274, thereby designated an aperture at the intersection of the row designated be the lit element 272 and the column designated by lit element 274.

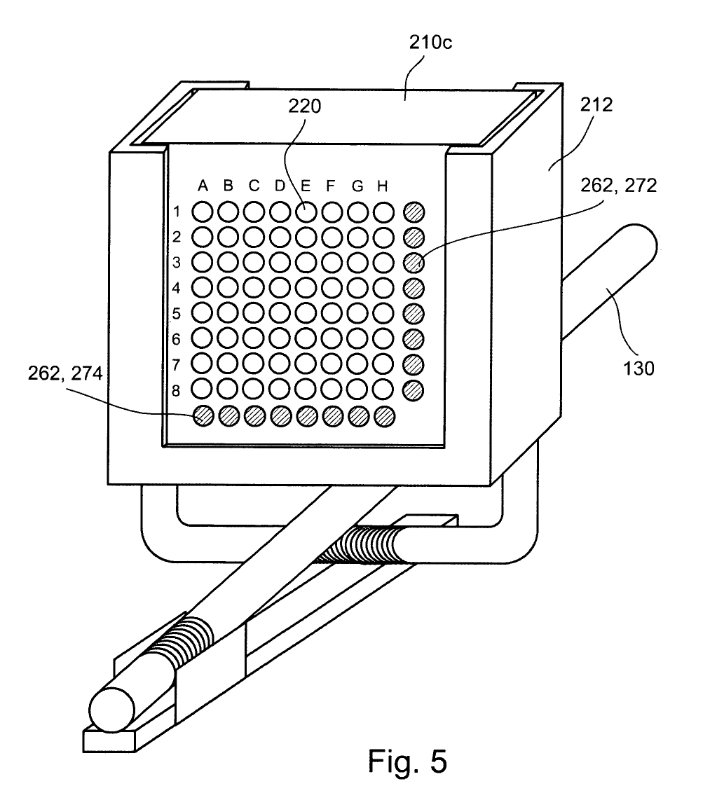

Attention is now drawn to FIG. 5, which is a simplified schematic of a template 210c designed for use attached to a frame 212, according to an embodiment of the present invention. In some clinical contexts it is preferable that guidance apparatus 200 be constructed in two parts, a first part comprising all or most of the relatively expensive (e.g. electronic) components, and a second part comprising template 210c supplied in sterile format and designed and constructed for one-time use. Alternatively, second part 210c in configured to be easily cleaned and sterilized for re-use, while the more delicate part containing most of the electronics is configured not to touch the patient or the probe and thus need not be sterilized. Template 210c is formed to fit and securely attach to a frame 212, which provides stability and a fixed position to template 210c with respect to other parts of guidance apparatus 200 and, thence, with respect to a patient. It is to be noted that FIG. 5 presents an exemplary means of attaching frame and template, but many alternative means of attaching frame to template are available.

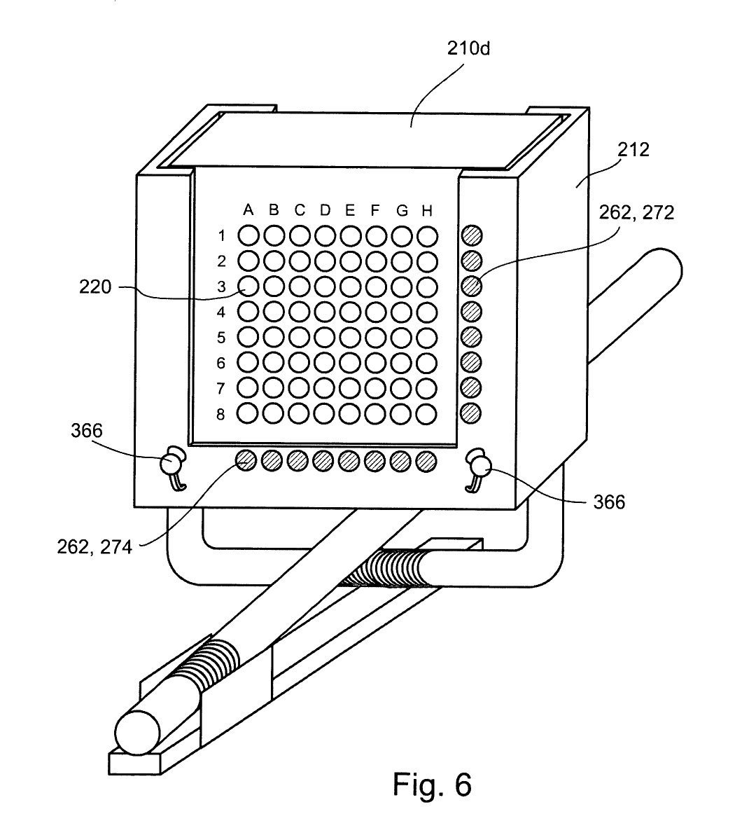

Attention is now drawn to FIG. 6, which is a simplified schematic of a template/frame combination utilizing a template 210d of simplified construction, according to an embodiment of the present invention. As may be seen in FIG. 6, potentially delicate (e.g. electric and electronic) elements may be installed on frame 212, enabling simplified construction of template 210d, which may then be constructed as a simple rectangular block comprising a plurality of apertures, constructed of a simple and homogeneous material such as metal or plastic. As such, template 210d may be subjected to sterilization procedures and is thus appropriate for repeated use, as opposed to template 210c designed for one-time use. In the exemplary embodiment shown in FIG. 6, user-directing output signals 250 are provided by paired lights 262 provided on frame 212 rather than on template 210d, and probe detection 350 is provided by cameras 366 and other components (not visible in this Figure) of image-interpretation detector 364.

In an exemplary embodiment of the invention, a template-add-on is provided which can fit on and/or be fixedly attached to an exiting template (e.g., using clamps) or frame. Optionally, a broad range of templates are supported by the add-on. Optionally, a calibration procedure is followed to match up the existing template positions and the add-on positions (and/or coordinate system).

Attention is now drawn to FIG. 7, which is a simplified schematic of an exemplary template comprising a variety of output devices and probe detection sensors, according to an embodiment of the present invention. FIG. 7 presents a template 210e. Template 210e is not intended to represent a realistic template embodiment, but rather is provided as a composite image showing additional views of various output devices and probe detection sensors mentioned hereinabove. Thus, aperture 220a is provided with an embedded light 256a operable to illuminate aperture 220a. Aperture 220b is provided with an off-plane light 256b operable to focus a light beam on aperture 220b, illuminating it. Aperture 220c is provided with colored lights 256c and 256d of differing colors. Light beam projection devices 260 are aimed to beam light towards probe bodies when those probes are inserted into selected apertures. Aperture 220d is provided with a magnetic sensor 352. Aperture 220e is provided with a microswitch 354. Aperture 220f is provided with a set of electric contacts 358 communicating with other components of an electrical detector 356.

Aperture 220g is provided with a probe insertion depth detector 270, operable to detect a depth to which an inserted probe 225 is inserted therethrough. In a preferred embodiment, depth detector 270 is an optical reader 271 operable to determine direction of movement of probe 225 within aperture 220f, and operable to count depth markings 273 provided on probe 225 as probe 225 is inserted into or withdrawn from aperture 220f, during which movement markings 273 pass in proximity to optical reader 271. In a particularly preferred embodiment, most or all apertures of a template 210 are provided with depth detectors 270, and depth detectors 270 are operable to provide probe depth information to controller 232. Controller 232 is thus enabled to provide probe depth information to a user through appropriate output devices 250 (such as through an image display) and is further enabled to provide insertion guidance and insertion feedback to a user, as taught in general hereinabove, thus enabling to guide and correct a user with respect to probe insertion depth as related to probe depth commands 224 as well as with respect to probe insertion placements as compared to probe placement commands 222.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A probe-insertion guiding apparatus comprising:
(a) a template which comprises a plurality of apertures sized and shaped to accommodate therapeutic probes inserted therethrough;
(b) a command receiver operable to receive a command specifying a template aperture through which a probe is to be inserted; and
(c) at least one of group consisting of:
(i) a first output device operable to output a signal serving to indicate which template aperture is specified by said received command; and
(ii) a probe detection module operable to detect when and where a probe is inserted in one of said plurality of apertures.

2. The apparatus of claim 1, comprising said first output device, and wherein said output signal is sensory signal perceivable by a user.

3. The apparatus of claim 1, comprising said first output device, and wherein said output signal is an electronic signal operable to control a servomechanism.

4. The apparatus of claim 3, wherein said electronically generated signal is a digital signal.

5. The apparatus of claim 3, wherein said electronic signal is an analog signal.

6. The apparatus of claim 3, wherein said electronic signal is communicated by a means selected from a group consisting of wired communication, wireless radio communication, optical communication, and infra-red communication.

7. The apparatus of claim 1, comprising both said first output device and said probe detection module.

8. The apparatus of claim 1, further comprising a feedback mechanism operable to inform a user whether or not a probe inserted by said user has been inserted in an aperture specified by a received command.

9. The apparatus of claim 1 comprising said first output device, and wherein said output device comprises at least one of a group consisting of:
(a) a lighting device operable to highlight a selected aperture;
(b) a pair of lighting devices operable to specify a selected aperture by highlighting a specific row of apertures and a specific column of apertures;
(c) a pair of light beams operable to highlight a portion of a probe positioned near or within a selected aperture;
(d) a LED highlighting module operable to indicate a selected aperture by illuminating selected LEDs, thereby creating a visual pattern which comprises two illuminated lines intersecting at said selected aperture;
(e) an auditory output device operable to emit a characteristic sound when a probe approaches a selected aperture;
(f) a voice output device operable to verbally identify a selected aperture; and
(g) an image output device operable to present to a user an image of said template on which an image of a selected aperture is highlighted.

10. The apparatus of claim 9, comprising a lighting device operable to use colored illumination to highlight a selected aperture.

11. The apparatus of claim 1 comprising said probe detection device, and wherein said probe detection device comprises at least one of a group consisting of:
(a) a magnetic detector;
(b) a microswitch;
(c) a current detector operable to detect a current in a circuit completed by passage of a probe body between electrical contacts;
(d) a voltage detector operable to detect a voltage in a circuit completed by passage of a probe body between electrical contacts; and
(e) an image-interpretation detector which comprises
(i) a camera operable create a real-time image of said template; and
(ii) image interpretation software operable to detect presence of a probe in or near a selected aperture by algorithmic interpretation of a real-time template image created by said camera.

12. The apparatus of claim 7, further comprising a feedback module which comprises a second output device and a controller operable to receive input from said probe detection device and to command said second output device to output a pre-defined first signal when said probe detection device reports insertion of a probe in an aperture specified by said received command.

13. The apparatus of claim 12, wherein said first output device and said second output device are a common output device.

14. The apparatus of claim 7, further comprising a feedback module which comprises a second output device and a controller operable to receive input from said probe detection device and to command said second output device to output a pre-defined second signal when said probe detection device reports insertion of a probe in an aperture different from that specified by said received command.

15. The apparatus of claim 12, wherein said controller is further operable to command said second output device to output a pre-defined second signal when said probe detection device reports insertion of a probe in an aperture different from that specified by said received command.

16. The apparatus of claim 12, wherein said second output device comprises one or more of a group consisting of:
(a) a generator of light;
(b) a generator of sound;
(c) a voice generator;
(d) an image display;
(e) an electronic signal generator operable to generate a signal detectable by a remote electronic receiver; and
(f) a control-signal generator operable to generate a signal directed to a remote controller operable to control activation of an inserted probe.

17. The apparatus of claim 1, further comprising at least one depth detector operable to detect depth of insertion of a probe within an aperture.

18. The apparatus of claim 17 wherein said depth detector is an optical reader operable to count markings on an inserted probe as said probe is inserted in an aperture.

19. The apparatus of claim 18, further operable to provide feedback to a user, which feedback provides information relating actual inserted depth of said inserted probe to a probe depth command received by said command receiver.

20. The apparatus of claim 1, further comprising a reusable frame to which said template is attachable, said frame and said template being so shaped and so positioned when said template is attached to said frame that a probe may traverse said apertures of said template while said template is attached to said frame.

21. The apparatus of claim 20, wherein said frame comprises at least one electrical component.

22. The apparatus of claim 20, wherein said template is designed for one-time use.

23. A method of guiding probe insertion by a user, comprising:
(a) indicating a next probe location to a user;
(b) detecting an indication that a probe was inserted into the location.
